(19) United States
(12) Patent Application Publication
(10) Pub. No.: US 2007/0208408 A1
(43) Pub. Date: Sep. 6, 2007

(54) Non-foreshortening sheaths and assemblies for use

(75) Inventors: Jan Weber, Maple Grove, MN (US); Thomas J. Holman, Minneapolis, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/368,913

(22) Filed: Mar. 6, 2006

(51) Int. Cl. A61F 2/06

(52) U.S. Cl. 623/1.11

(57) ABSTRACT

A catheter assembly comprises a catheter shaft having a device receiving portion, an expandable balloon disposed about the device receiving portion, and a sheath. The sheath has an outer surface and an inner surface defining an inner lumen. The outer surface and inner surface has a sheath wall extending there between. The outer surface has slits therethrough which extend into the sheath wall. The sheath has an expanded state and an unexpanded state such that the sheath maintains the substantially same longitudinal length in the expanded state and the unexpanded state.

### CROSS-REFERENCE TO RELATED APPLICATIONS

Not Applicable

### STATEMENT REGARDING FEDERALLY SPONSORED RESEARCH

Not Applicable

### FIELD OF THE INVENTION

In some embodiments this invention relates to implantable medical devices, their manufacture, and methods of use. Some embodiments are directed to delivery systems, such as catheter systems of all types, which are utilized in the delivery of such devices.

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to herein as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels, for example, as the bifurcation in the mammalian aortic artery into the common iliac arteries. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such.

The art referred to and/or described above is not intended to constitute an admission that any patent, publication or other information referred to herein is “prior art” with respect to this invention.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In at least one embodiment, an inventive sheath can comprise an inner surface defining an inner lumen and an outer surface with a sheath wall extending between the outer surface and the inner surface, the outer surface having one or more slits which extend into the sheath wall. The sheath has an expanded state and an unexpanded state; the sheath maintaining the substantially same longitudinal length in the expanded state and the unexpanded state. In this application a “slit” refers to any slit, hole, port, opening, indentation, or other such surface features.

In at least one embodiment, where at least the outer surface defines a one or more slits therein, the slit(s) can be arranged randomly or in accordance with a desired pattern or patterns.

At least some embodiments of the present invention are directed to stent delivery systems which have a rotatable sheath or other mechanism for imparting rotatability to the pre-delivered stent or other implantable medical device.

In at least one embodiment, the slits can be constructed and arranged such that at least the outer surface of the sheath defines cells that are characterized as being bi-stable.

In at least one embodiment, the bi-stable sheath has only two stable configurations, namely the expanded state and the unexpanded state. In the unexpanded state the sheath can be rotatably disposed about a balloon or other expansion member prior to its expansion, whereas in the expanded state the balloon has been expanded to an increased diameter, thus increasing the diameter of the sheath to its expanded state. In at least one embodiment, when the sheath is in the expanded state the cells have an expanded circumferential width which is greater than the circumferential width of the cells in the unexpanded state. In at least one embodiment, the length of the sheath in the expanded state is substantially equal to the length of the sheath in the unexpanded state.

In at least one embodiment, the slits form bi-stable cells having a first state wherein the cell exerts a force toward an expanded stable configuration and a second state wherein the cell exerts a force toward an unexpanded stable configuration. In at least one embodiment, compressive force applied to the sheath is resisted until a transformation point is reached, before the transformation point is reached the cells and sheath tends to the first state, after the transformation point is reached the cells and sheath tends to the second state.

In at least one embodiment, the slits extend only partially through the sheath wall.

In at least one embodiment, the slits only extend through 80% to 90% of the thickness of the sheath wall.

In at least one embodiment, the slits can extend entirely through the sheath wall, defining an opening through the wall that extends from the outer surface to the inner surface.

In at least one embodiment, the sheath can be incorporated into a bifurcated stent delivery system.

In at least one embodiment, the sheath can be formed of a plastic.

In at least one embodiment, the cells of the sheath do not deform plastically when going from one stable configuration to another stable configuration.

In at least one embodiment, the sheath has an unexpanded state and an expanded state such that in the expanded state the cells are in an opened bi-stable configuration and in the unexpanded state the cells are in a closed bi-stable configuration.

In at least one embodiment, the sheath can be at least partially formed of metal.

In at least one embodiment, the cells of the sheath can have hinges which deform plastically when going from one stable configuration to another stable configuration.

In at least one embodiment, the cells of the sheath do not deform plastically when going from one stable configuration to another stable configuration.

In at least one embodiment, an additional layer constructed of material substantially weaker than that of the sheath can be applied to the interior of the sheath or to the exterior of the sheath.

In at least one embodiment, any of the above sheaths can be placed on a catheter assembly having a device receiving portion and an expandable balloon disposed about the device receiving portion.

In at least one embodiment, a stent is disposed about the sheath such that in the unexpanded state the stent remains engaged to the sheath and in the expanded state the stent is unengaged to the sheath.

In at least one embodiment, a method for manufacturing any of the above sheaths can comprise:
providing a tubular member; and
cutting at least one slit at least partially into the outer surface of the tubular member using a laser.

In at least one embodiment, an excimer laser is used. In at least one embodiment, the laser can operate in pulses and remove a portion of material with each pulse.

In at least one embodiment, the slits can have a pattern produced by using a computer numerical control (CNC) mechanism which can rotate and move under a laser beam in a fixed position.

In at least one embodiment, the slits can have a pattern produced by using holographic diffraction or a template.

In at least one embodiment, a method for manufacturing any of the above sheaths can comprise molding a polymer into a sheath with slit patterns.

These and other embodiments of the invention are pointed out with particularity in the claims annexed hereto and forming a part hereof. However, for additional understanding of the invention, its advantages and objectives obtained by its use, reference should be made to the drawings which form a further part hereof and the accompanying descriptive matter, in which there is illustrated and described various embodiments of the invention.

### BRIEF DESCRIPTION OF THE SEVERAL VIEWS OF THE DRAWING(S)

A detailed description of the invention is hereafter described with specific reference being made to the drawings.

FIG. 1 illustrates a partial side view of an unexpanded sheath having slits.

FIG. 1a illustrates a partial side view of an expanded sheath having slits.

FIG. 2 is a perspective view of a sheath in the unexpanded configuration.

FIG. 3 is a perspective view of a sheath in the expanded configuration.

FIGS. 4A-B illustrate the concept of the bi-stable characteristic.

FIG. 4C is a cross-sectional view of a bi-stable embodiment.

FIGS. 5A-B is a top view of an embodied cell having joints.

FIGS. 6A-B is a top view of an embodied cell having joints.

FIGS. 7A-B is a top view of multiple embodied cells attached together.

FIG. 8 is a cross-sectional view of a sheath having an interior and exterior layer applied.

FIGS. 9A-B is a cross-sectional side view of the sheath in conjunction with a balloon and stent.

FIG. 10 is a cross-sectional side view of a catheter assembly.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

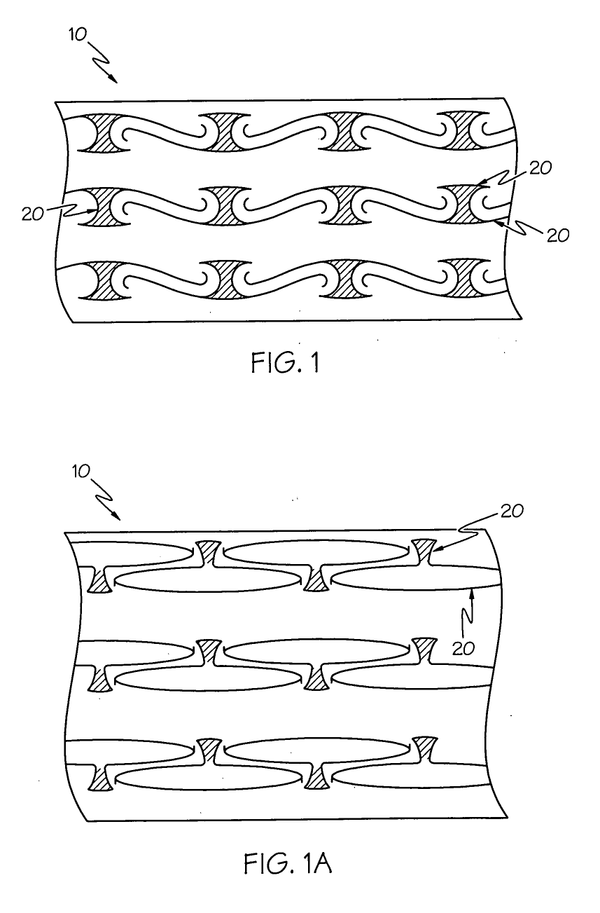

Referring generally to FIG. 1, a partial side view of an unexpanded sheath 10 having slits 20 is shown. In the unexpanded state the slits 20 as shown cover only a small portion of the surface of sheath 10. This improves the sheath rotation about a balloon by lessening the chance of balloon folds getting stuck in the slits 20. In the expanded state, illustrated in FIG. 1a, the slits 20 widen and the circumference of the sheath 10 increases.

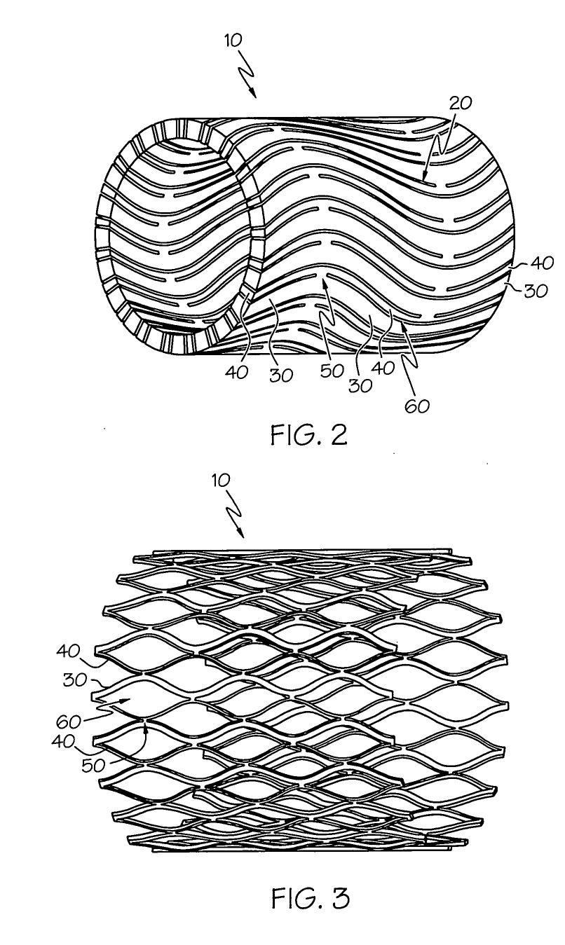

FIG. 2 illustrates a perspective view of a partial sheath 10 in an unexpanded configuration and having slits 20. This slit pattern has thicker portions 30 and thinner portions 40 connected by connecting portion 50. In the unexpanded configuration the cells 60 (only a partial cell shown in FIG. 2) are in a first stable configuration. As shown here, an entire cell is not shown; if the view of the sheath was extended the thinner portions 40 and the thicker portions 30 would connect at another connecting portion 50 as shown in FIG. 3 which shows the sheath 10 of FIG. 2 in the expanded configuration. FIG. 3 is of a smaller scale than FIG. 2 and illustrates an extended sheath portion which has multiple complete cells having connectors 50. connecting the thinner portions 40 and the thicker portions 30. If the cells are bi-stable, then as expansive forces are applied to the sheath 10 the thicker portions 30 and the thinner portions 40 will move away from one another such that the slit 20 widens and the cell 60 expands thereby increasing the area of the cell. The bi-stable cells have a transition point. The cell configuration about the transition point determines which stable configuration the bi-stable cell will exert a force toward. In some embodiments, if compressive or expansive force is applied to a cell when the size of the cell is smaller than the transition point size then the cell will exert a force tending toward a closed or unexpanded configuration, resisting expansion; if compressive or expansive force is applied to a cell when the size of the cell is larger than the transition point size then the cell will exert a force tending toward an open or expanded configuration, resisting compression.

A cell is “bi-stable” when it has two or more discrete stable configurations, including a first stable configuration with a first circumferential width and a second stable configuration with a second larger circumferential width, such that when a force is applied to the cell, the cell will tend to or exert a force in the direction of one of the discrete configurations. In at least one embodiment the cell will tend to one or another of the discrete configurations depending on whether the cell has been compressed beyond a transition point. If the cell has been compressed beyond the transition point the cell will tend toward a closed configuration; if the cell has not been compressed to the transition point the cell will tend toward an open configuration. In at least one embodiment the sheath is in the unexpanded state when the cells are in the closed configuration and in the expanded state when the cells are in the open configuration. Cells can be designed in such a way that there are cells with a low energy threshold in passing the transition point and other cells with a higher energy threshold in passing the transition point. In at least one embodiment, the thickness of the thin element 40 or the length of the individual cells can be changed. In at least one other embodiment, multiple materials can be combined in a stent design with high and low stiffness (e.g. in the case of polymeric stents). In at least one embodiment, individual elements (40) can be stiffened in the finished stent by laser shot peening. It should be noted that in some expanded states some cells may remain closed while in some unexpanded states some cells may be in the open configuration. It should be further noted that within one stent design one can incorporate cells with different transition points such that the stent opens in a systematic manner. Such a design can allow for a very predictable expansion of the stent.

In some embodiments the sheath 10 behaves in a partly balloon expandable and a partly self expanding manner. Balloon expansion can be used up to the transition point. Upon reaching the transition point the sheath then self expands as it moves to the expanded bi-stable state. This allows a balloon having a smaller expansion diameter to be used as the balloon needs to only expand the sheath to the transition point. In addition other actuators, such as electro-active polymers, can be used which have a limited expansion range, but enough expansion such that if a sheath is mounted upon them the sheath can reach the transition point, whereupon it will self expanded to the expanded stable state.

In some embodiments, the sheath has bi-stable cells and conventional cells. This can help a physician adapt the sheath diameter to match the vessel diameter precisely.

When forming the sheath, the pattern cut out can be either for the expanded or the unexpanded bistable configuration. In at least one embodiment, the expanded state/configuration is used. In the expanded state the various electropolishing methods can be more readily used. In addition, if formed in the expanded state one less deformation step is required when crimping the sheath to a balloon.

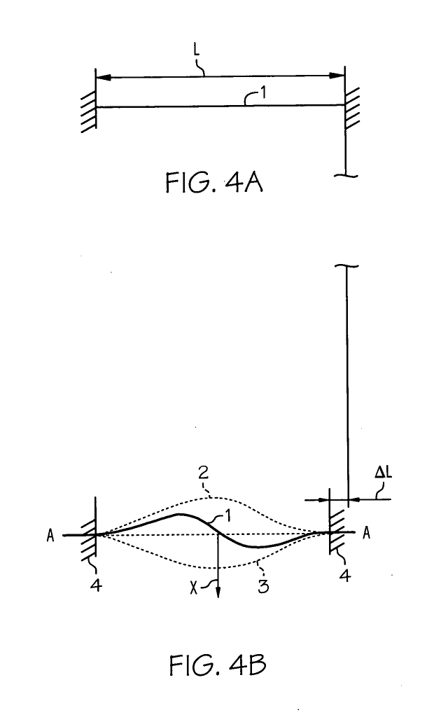

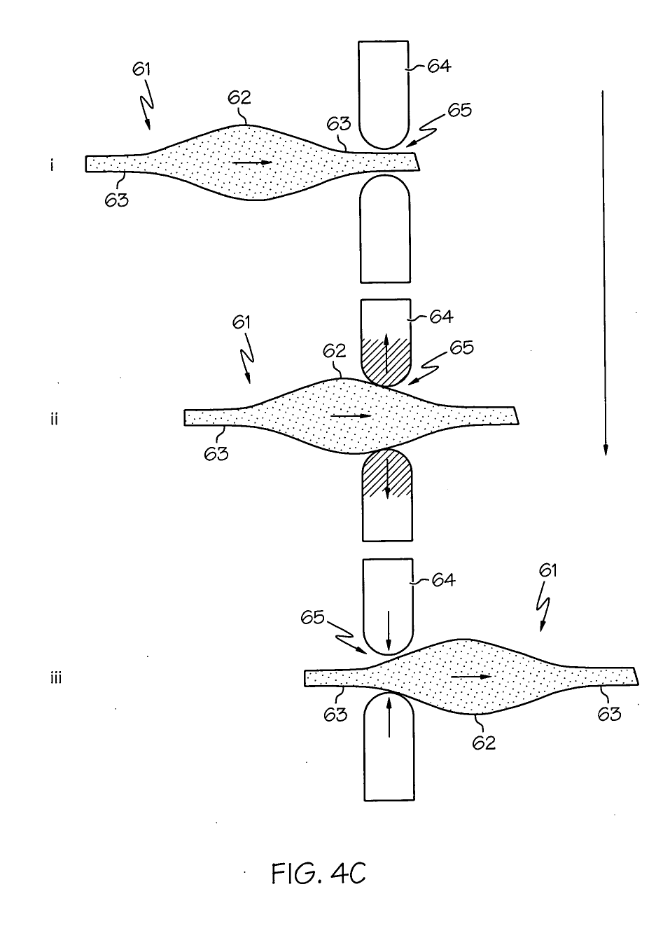

FIGS. 4A-4B illustrated an embodied bi-stable characteristic. FIG. 4A shows a rod 1 with a length L, which is compressed in its axial direction by a distance ΔL and reaches its buckling stress. Then the central part of the rod will bend out in a sidewards direction, either to position 2 or 3 (dashed lines in FIG. 4B). When the axial displacement L of the ends of the rod is held stable by external clamps 4, it is possible to move the central section of the rod between the two stable positions 2 and 3. This movement is in a direction X, perpendicular to the original length axis A-A of the rod. All positions between the stable positions 2 and 3 are unstable. As shown here, the transition point is the point at which half of the rod 1 is on one side of the axis A-A and the other half is on the other side of axis A-A. FIGS. 4A-4B illustrate an embodiment of the bi-stable characteristic. Many different configurations for a bi-stable cell can be drawn from this illustration. This basic bi-stable characteristic is also evident in the other figures in this application.

It should further be noted that the expansion ratio of a stent is determined by the cell length L and the amplitude 5 as shown in FIGS. 4A and 4B. While the cell lengths shown in FIG. 2 are symmetrical, by reducing the cell length L from the proximal end of the stent or sheath to the distal end a tapered stent or sheath in the expanded state can be produced while being fully straight in the compressed state.

An example of constructing a delivery system having a bi-stable stent or sheath is given below:

A stainless steel stent or sheath (stent) is cut out of a 1.2 mm diameter tube with wall thickness of 100 micrometer. One method of cutting is “water jet” cutting. The pattern being used is as drawn in FIG. 2 with the thinner portions 40 being about 50 micrometers and the thicker portions being about 120 micrometers. The cell length L is about 2 mm and the stent is about 5 cell lengths long. Circumferentially there are 20 cells. The deflection 5 (as illustrated in FIG. 4B) from the centerline is about 10% of the length L, about 200 micrometers. Upon expansion the circumference of the stent will increase by about 8.0 mm (20×2×200 micrometer). The total circumference is 8.0 mm+20(170 micrometer)=10.4 mm; thus, the diameter of the expanded stent is about 3.3 mm. The expanded stent is placed on a 3.2 mm diameter balloon and compressed by a conventional crimper to collapse to the lower bistable state. However, the folded balloon is made with a profile of 1.3 mm such that the stent will apply a residual crimp force while on the balloon. After fully deploying the balloon, the stent will be forced to 3.2 mm and because of the residual outward force, the stent will have a greatly reduced recoil or no recoil in contrast to many conventional stents.

Another bi-stable embodiment is shown in cross-section in FIG. 4C. In the first frame (i) of FIG. 4C element 61 having a wide portion 62 and narrow portions 63 is shown entering into the center opening 65 of ring 64 which is stable. Ring 64 may be constructed of rubber or metal. As shown in second frame (ii), when element 61 is further advanced into the center opening 65 the wide portion 62 forces the center opening 65 to expand. As shown in third frame (iii), when the wide portion 62 passes through the center opening 65 the opening begins to contract as the ring 64 begins to return to the stable position of the first frame (i). During the contraction, the ring 64 exerts a force on element 61 to continue moving it forward until the ring 64 is again stable. This embodiment may be used in an interlocking stent system or the like. Element 62 might be an extending portion of a stent or stent graft that locks into a ring like portion 64 of another stent or stent graft.

FIG. 3 is a broadened view of the sheath of FIG. 2 in the expanded state. Here, numerous complete cells 60 are shown. The thicker portions 30 are attached to the thinner portions 40. Upon expansion, the thinner portions move due to the expansive force and the ends of the thicker portions 30 in some embodiments can be characterized as acting as the external clamps 4 shown in FIG. 4B. The thicker portions 30 maintain the substantially same shape and configuration during expansion thus substantially eliminating foreshortening of the sheath 10; the thinner portions 40 move from the stable closed configuration of FIG. 2 to the stable open configuration of FIG. 3 thus resulting in expansion of the sheath 10.

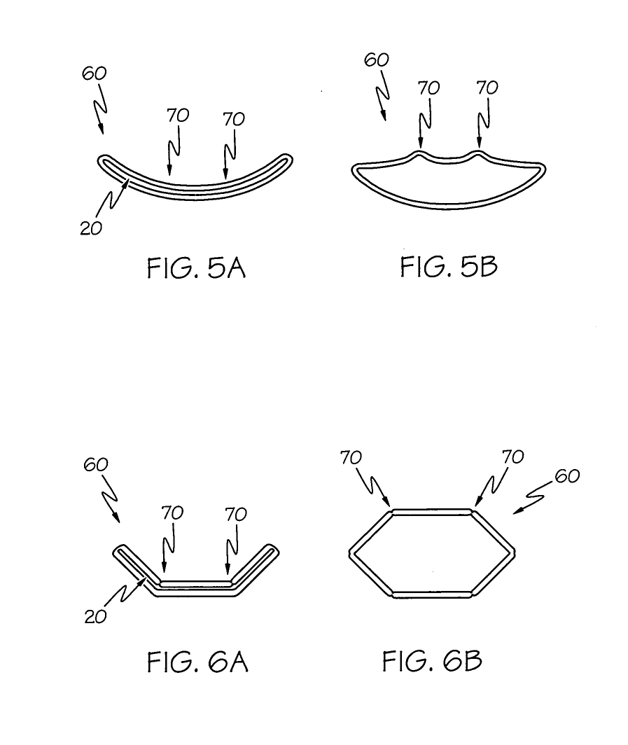

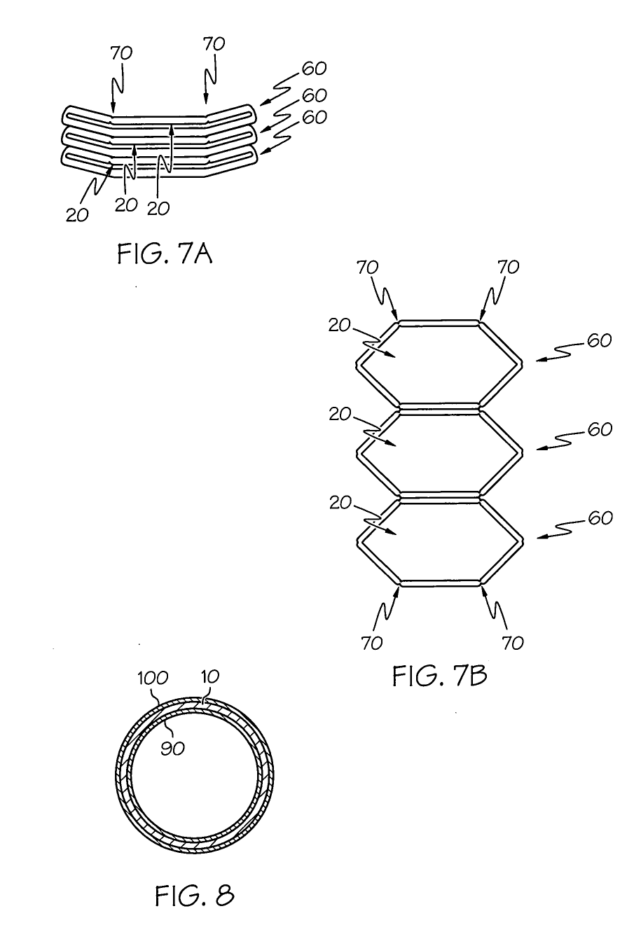

FIGS. 5A-7B illustrate various other configurations of bi-stable cells. The bi-stable cells 60 in these configurations have bending joints 70. These joints 70 can be constructed of a more ductile material than the other portions of the cell 60, or the material may be thinner. FIGS. 7A-7B illustrate multiple cells 60 having slits 20 and joints 70. These multiple cells can comprise a sheath or a portion of a sheath.

In some embodiments, the sheath 10 can have a double layer or a tri-layer construction as shown in cross-section in FIG. 8. In at least one embodiment a bi-stable sheath portion 10 as described above can have an additional interior layer 90 and/or exterior layer 100 applied. The interior and exterior layers can be constructed of a material substantially weaker than the bi-stable sheath portion 10 (e.g. a thin silicon rubber material mounted on a metallic sheath or stent portion 10). Thus, when sheath portion 10 expands to an expanded cell configuration, layers 90 and 100 will not interfere with the non-foreshortening nature of sheath portion 10. It should be noted that interior layer 90 can be constructed from the same piece of material as the sheath 10 itself; the interior layer 90 can be formed by cutting slits into the sheath 10 which do not go through the entire sheath wall, thereby leaving an interior layer uncut.

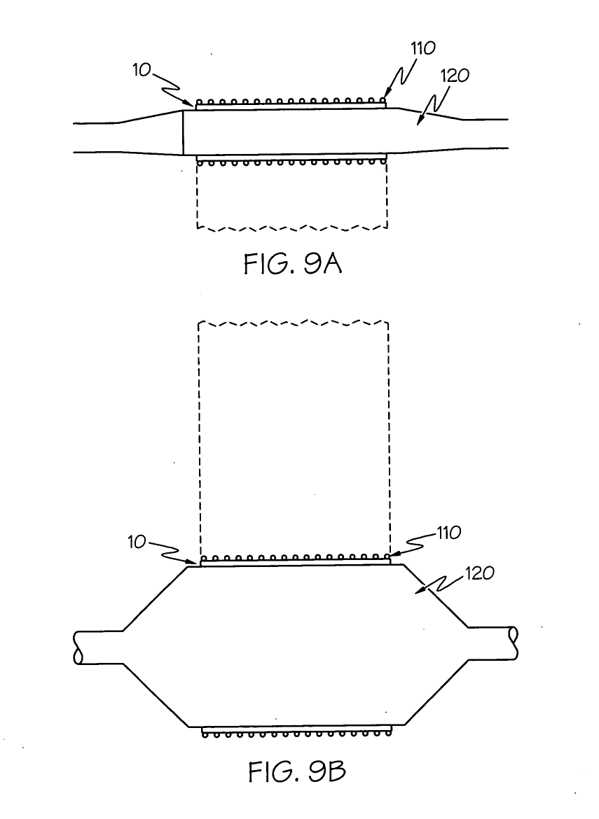

FIGS. 9a-b illustrate the non-foreshortening nature of the sheath 10. In FIG. 9a a stent 110 is disposed about non-foreshortening sheath 10 in an unexpanded state. The sheath 10 and stent 110 disposed about a balloon 120. When the balloon 120 expands into an expanded state, the sheath 10 maintains the substantially same length allowing the stent 110 to also expand without foreshortening. The assembly of FIGS. 9a-b can be used at a bifurcated site as well as a non-bifurcated site. For treatment at a bifurcated site, the sheath 10 can be constructed to rotate about the balloon. In some embodiments, a stent 110 placed about the sheath 10 can rotate about the sheath.

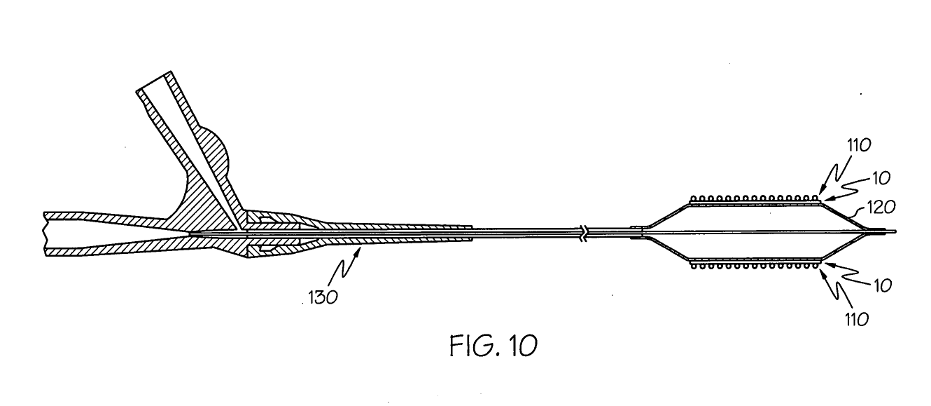

The stent 110 is shown in FIG. 10 disposed about the sheath 10 as a part of the catheter assembly 130. The catheter assembly can be any sort of catheter assembly including a bifurcated stent delivery system for delivering a stent 110 to a bifurcation. A bifurcated stent 110 can have a trunk with two branches. In at least one embodiment, the trunk and one of the branches have longitudinal axes that are parallel to one another or even share an identical longitudinal axis.

In at least one embodiment, the bifurcated stent delivery system is configured to advance the stent 110 along guide wires to a bifurcation site where it is positioned within the primary vessel to extend across the secondary vessel (or side branch) of the bifurcation. When expanded, one of the branches of the bifurcated stent extends into the secondary vessel and one of the branches extends into the primary vessel in order to provide support for the bifurcation site.

The sheath 10 is disposed about the balloon 120. The sheath can be constructed of material that allows the sheath 10 to rotate about the balloon 120. When delivering a medical device such as a stent to a treatment site rotation of the medical device within the vessel during advancement to the treatment site is often desirable. In instances where the medical device is configured for deployment at a bifurcation of vessels it is especially desirable to impart rotatability to the medical device prior to its delivery in order to properly align a side opening or branch of the device with the side branch vessel of the bifurcation.

Some stent delivery systems have been developed, which impart rotation to the stent retaining region of the catheter by positioning a unique rotatable sheath underneath the stent prior to its delivery. Such systems and sheathes are described in a variety of references including: U.S. patent application Ser. No. 10/375,689, filed Feb. 27, 2003 and U.S. patent application Ser. No. 10/657,472, filed Sep. 8, 2003 both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; U.S. patent application Ser. No. 10/757,646, filed Jan. 13, 2004 and entitled Bifurcated Stent Delivery System; and U.S. patent application Ser. No. 10/784,337, filed Feb. 23, 2004 and entitled Apparatus and Method for Crimping a Stent Assembly; U.S. patent application Ser. No. 10/863,724, filed Jun. 8, 2004 and entitled Bifurcated Stent Delivery Sheath, the entire content of each being incorporated herein by reference.

In at least one embodiment, the sheath is constructed of any suitable material, such as polyesters and copolymers thereof such as those sold including polyalkylene terephthalates such as polyethylene terephthalate (PET) and polybutylene terephthalate (PBT) available under the tradename of EKTAR® available from Eastman Chemical Co. in Kingsport, Tenn., polycyclohexylene terephthalate (PCT); poly(trimethylene terephthalate) (PTT), PCTG and poly(cyclohexanedimethanol-co-ethylene terephthalate) (PETG) copolyesters available under the tradename of EASTAR® available from Eastman Chemical Co., PCTA available under the tradename of DURASTAR® available from Eastman Chemical Co., poly(ethylene naphthalate) (PEN) polyester available from DuPont in Wilmington, Del. under the tradename of TEONEX®; and so forth; polyester elastomers (PEELs); polyamides such as amorphous nylon and nylon 12 such as those available from Elf Atochem under the tradename of CRISTAMID® and copolymers thereof such as GRILAMID® TR-55-LX nylon 12 polyether-block-amide available from EMS-American Grilon in Sumter, S.C.; polyetherimides available from GE Plastics under the tradename of ULTEM®; polystyrene and expandable polystyrene (EPS); acrylonitrile-butadiene-styrene (ABS); styrene-acrylonitrile (SANs); polyphenylene sulfide (PPS); polyphenylene oxides (PPO); interpolymers of PPO and EPS; polyetherketones (PEEK); polyolefins such as polyethylenes and polypropylenes including low, medium and high densities such as HDPE available under the tradename of ALATHON® from Equistar Chemicals; amorphous polyolefins; polyether-block-amides such as those sold under the tradename of PEBAX® available from Elf Atochem; polyimides; polyurethanes; polycarbonates; polyethers; silicones; as well as any copolymers thereof. The above list is intended for illustrative purposes only, and is not intended to limit the scope of the present invention. One of ordinary skill in the art has knowledge of such polymeric materials. Poly-L Lactic Acid (PLLA) or some other bio-degradable material may also be used.

In order to ensure that the sheath 10 is rotatable about a balloon, even with a stent crimped on to the sheath and the catheter is being advanced through the a body, the sheath may be constructed of a variety of low friction materials such as PTFE, HDPE, etc. In at least one embodiment the sheath is at least partially constructed of a hydrophilic material, such as hydrophilic polymers such as; TECOPHILIC® material available from Thermedics Polymer Products, a division of VIASYS Healthcare of Wilmington, Mass.; TECOTHANE®, also available from Thermedics Polymer Products; hydrophilic polyurethanes, and/or aliphatic, polyether-based thermoplastic hydrophilic polyurethane; and any other material that provides the sheath 10 with the ability to rotate freely about the balloon 120 when in the “wet” state, such as when the catheter is exposed to body fluids during advancement through a vessel. Suitable sheath materials may also provide the sheath with rotatability in the “dry”, or pre-insertion, state, but with the application of a greater amount of force than when in the wet state, such materials are referred to herein as being tecophilic.

The sheath 10 can be at least partially constructed from tecophilic material which provides the sheath with the ability to rotate freely about the balloon when in the “wet” state. The tecophilic sheath is also capable of rotation in the “dry” state, but with the application of a greater amount of force than when in the wet state. The composition of the sheath 10 material, whether a single or multiple layer may include essentially any appropriate polymer or other suitable materials. Some example of suitable polymers include Hydrophilic Polyurethanes, Aromatic Polyurethanes, Polycarbonate base Aliphatic Polyurethanes, Engineering polyurethane, Elastomeric polyamides, block polyamide/ethers, polyether block amide (PEBA, for example available under the trade name PEBAX), and Silicones, Polyether-ester (for example a polyether-ester elastomer such as Arnitel available from DSM Engineering Plastics), Polyester (for example a polyester elastomer such as Hytrel available from Du Pont), or linear low density polyethylene (for example Rexell).

The sheaths described above can include a stent for supporting a vessel lumen. In some embodiments, the sheath itself can function as a stent and support a vessel lumen. In some embodiments, stents can replace the sheaths as described above. In some embodiments the stents are self expanding or balloon expandable. In some embodiments the stent may be constructed of Nitinol or other shape memory metal, titanium, stainless steel, Elgiloy, NP35N, Hastelloy, or other alloyed metals. Shape memory polymers such as cross linked polyurethanes, polynorbornene, poly dimethacrylate, and biodegradable shape memory polymers such as oligo(ε-caprolactone)diol.

In some embodiments, the stent could be pre-stressed to a plastic state and formed inside the body. This can allow the system to be built inside of the legion or affected area.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The sheaths and/or stents as described above can be used in both gastro systems and vascular systems.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of various embodiments of the invention. Those skilled in the art may recognize other equivalents to the specific embodiment described herein which equivalents are intended to be encompassed by the claims attached hereto.

1. A catheter assembly comprising:
a catheter shaft having a device receiving portion;
an expandable balloon disposed about the device receiving portion; and
a sheath disposed about the balloon, the sheath having:
an inner surface defining an inner lumen;
an outer surface, the outer surface and inner surface having a sheath wall extending there between;
the outer surface having slits therethrough which extend into the sheath wall, the sheath having an expanded state and an unexpanded state, the unexpanded state having a smaller diameter than the expanded state, the sheath maintaining the substantially same longitudinal length in the expanded state and the unexpanded state.

2. The catheter assembly of claim 1 wherein the slits extend only partially through the sheath wall.

3. The catheter assembly of claim 2 wherein the slits only extend through 80% to 90% of the thickness of the sheath wall.

4. The catheter assembly of claim 1 wherein the slits extend through the sheath wall and inner surface.

5. The catheter assembly of claim 1 in combination with a stent.

6. The catheter assembly of claim 1 wherein the sheath is rotatable about the balloon.

7. The catheter assembly of claim 1 wherein a stent is disposed about the sheath, in the unexpanded state the stent remaining engaged to the sheath, in the expanded state the stent being unengaged to the sheath.

8. The catheter assembly of claim 1 wherein the sheath has an unexpanded state and an expanded state, in the expanded state the cells being in an opened bi-stable configuration, in the unexpanded state the cells in a closed bi-stable configuration.

9. The catheter assembly of claim 1 wherein an additional layer constructed of material substantially weaker than that of the sheath is applied to the interior of the sheath or to the exterior of the sheath.

10. The catheter assembly of claim 1 wherein the sheath is formed of a plastic.

11. The catheter assembly of claim 10 wherein the cells of the sheath do not deform plastically when going from one stable configuration to another stable configuration.

12. A catheter assembly comprising:
a catheter shaft having a device receiving portion;
an expandable balloon disposed about the device receiving portion; and
a sheath, the sheath having:
an inner surface defining an inner lumen;
an outer surface, the outer surface and inner surface having a sheath wall extending there between;
the outer surface having slits therethrough which extend into and are defined by the sheath wall, the slits forming bi-stable cells, the bi-stable cells having a first state wherein the cell exerts a force toward an expanded stable configuration and a second state wherein the cell exerts a force toward an unexpanded stable configuration, the first state and second state such that compressive force applied to the sheath is resisted until a transformation point is reached, before the transformation point is reached the cells and sheath tends to a stable expanded state, after the transformation point is reached the cells and sheath tends to a stable unexpanded state.

13. The catheter assembly of claim 12 wherein the sheath maintains the substantially same longitudinal length during expansion of the sheath from an unexpanded state to an expanded state.

14. The catheter assembly of claim 12 wherein the cells are stable in only two positions

15. A catheter sheath comprising:
a tubular member having an inner surface defining an inner lumen and an outer surface, the outer surface and inner surface having a sheath wall extending there between;
the outer surface having slits therethrough which extend into the sheath wall, the sheath having an expanded state and an unexpanded state, the unexpanded state having a smaller diameter than the expanded state, the sheath maintaining the substantially same longitudinal length in the expanded state and the unexpanded state.

16. The catheter sheath of claim 15 wherein the slits form bi-stable cells.

17. The catheter sheath of claim 15 wherein an additional layer constructed of material substantially weaker than that of the sheath is applied to the interior of the sheath and to the exterior of the sheath.

18. The catheter sheath of claim 15 wherein the slits extend through the sheath wall and inner surface.

19. The catheter sheath of claim 15 in combination with a bifurcated stent delivery system, the bifurcated stent delivery system having an expandable balloon disposed about a catheter shaft, the sheath disposed about the balloon, a bifurcated stent disposed about the sheath, the bifurcated stent having a trunk and at least two branches.

20. The catheter sheath of claim 15 in combination with a catheter balloon, the sheath rotatable about the catheter balloon.
